(19) United States
(12) Patent Application Publication
(10) Pub. No.: US 2015/0004599 A1
(43) Pub. Date: Jan. 1, 2015

(54) REAL-TIME DNA-BASED IDENTITY SOLUTION

(71) Applicant: VERIZON PATENT AND LICENSING INC., Basking Ridge, NJ (US)

(72) Inventor: John S. PEREZ, Tampa, FL (US)

(21) Appl. No.: 13/929,704

(22) Filed: Jun. 27, 2013

(51) Int. Cl. G01N 33/53

(52) U.S. Cl. CPC G01N 33/5308
USPC 435/6.11; 436/94; 436/501; 422/69; 435/287.2

(57) ABSTRACT

Verifying a user's identity in real-time using DNA-based information includes obtaining a chromosomal DNA sample from a user, and causing a chromosome of the DNA sample to become mounted in a DNA sampler. The DNA sampler includes a linear array of capacitors in which pairs of capacitor plates are disposed end-to-end along a linear gap. The chromosome mounted in the DNA sampler is disposed in the linear gap with the pairs of capacitor plates located along a length of the chromosome. A chromosomal signature of the chromosome is obtained by measuring, for each pair of capacitor plates, an electrical property at the pair of capacitor plates. The electrical property can include one of capacitance, resistance, or conductance. A determination is made as to whether the obtained chromosomal signature is associated with the user by comparing the obtained chromosomal signature to stored chromosomal signatures of chromosomes of the user.

### BACKGROUND

In order to maintain the security of data and computer systems, and in order to combat electronic identity fraud, users are required to provide identity credentials as part of logging into computer systems. In general, the identity credentials are a username and password pair. More advanced identity solutions rely on alternative or additional means of identification including identity cards or tokens, facial recognition, fingerprint or iris scanners, or the like. Multi-factor identity solutions combine two or more identity solutions together to further increase the security afforded by the solution. In all cases, the identity credentials entered by or obtained from the user are compared to stored identity credentials for the user, and the user is either granted or not granted access to the data or computer system dependent on the result of the comparison.

Deoxyribonucleic acid (DNA) is present in all persons' cells, and stores a sequence of genetic codes that can be used to identify each person. Solid state electronic technologies have been developed for sequencing DNA. Such electronics are designed to determine the genetic sequence of codes stored in DNA strands. The electronics, however, cannot determine the genetic sequence alone. Instead, highly trained personnel have to use time consuming and complex DNA amplification and sequencing methods prior to determining the sequence. As a result, the cost of DNA identity solutions is high. Additionally, DNA sequencing cannot generally be used as part of an automated or real-time identity solution.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIGS. 1A and 1B are diagrams illustratively showing a chromosome.

FIG. 2 is a high-level functional block diagram of a device operative to obtain a chromosomal signature of a chromosome using an array of capacitors.

FIGS. 3A-3C are high-level functional block diagram of DNA samplers each operative to bind a chromosome and obtain a chromosomal signature of the chromosome.

FIG. 4 is a flow diagram illustratively showing steps of a method for verifying a user's identity in real-time using DNA-based information about the user.

FIGS. 5A and 5B are high-level functional block diagram of DNA samplers operative to bind pluralities of chromosomes and obtain chromosomal signatures of the chromosomes.

FIGS. 6A-6C are high-level functional block diagram of additional DNA sampler architectures.

FIGS. 7A-7D are high-level functional block diagram of structures including DNA samplers operative to obtain chromosomal signatures of chromosomes.

FIG. 8 is a high-level functional block diagram of a system of networks/devices that use chromosomal signatures for authentication.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

The various methods and systems disclosed herein relate to real-time DNA-based identity solutions, such as methods and systems for verifying a user's identity in real-time using DNA-based information about the user. In particular, the methods and systems include measuring an electrical property at locations along the length of a chromosome of the user, and determining whether the sequence of measurements matched a stored sequence for the user.

A method includes obtaining a chromosomal DNA sample from a user, and processing the chromosomal DNA sample in order to cause a chromosome of the DNA sample to become mounted in a DNA sampler. The method further includes controlling the DNA sampler to obtain a chromosomal signature of the chromosome from the DNA sampler, and determining whether the obtained chromosomal signature is associated with the user by comparing the obtained chromosomal signature to stored chromosomal signatures of chromosomes of the user. The DNA sampler includes a linear array of capacitors in which pairs of capacitor plates are disposed end-to-end along a linear gap. A chromosome mounted in the DNA sampler is disposed in the linear gap with the pairs of capacitor plates located along a length of the chromosome. The obtaining of the chromosomal signature includes measuring, for each pair of capacitor plates of the linear array having a portion of the chromosome disposed therebetween, an electrical property at the pair of capacitor plates. The electrical property can include one of capacitance, resistance, or conductance.

The DNA sampler includes a linear array of capacitors in which pairs of capacitor plates are disposed end-to-end along a linear gap. A chromosome mounted in the DNA sampler is disposed in the linear gap with the pairs of capacitor plates located along a length of the chromosome. The DNA sampler includes at least one binding trap structure, wherein the binding trap structure is disposed in alignment with the linear gap and is operative to bind with a centromere or a telomere of a chromosome mounted in the DNA sampler, and thereby fix the chromosome in the linear gap. The DNA sampler further includes circuitry coupled to each pair of capacitor plates and operative to obtain a chromosomal signature of a chromosome mounted in the DNA sampler by measuring, for each pair of capacitor plates of the linear array having a portion of the chromosome disposed therebetween, an electrical property at the pair of capacitor plates. The electrical property can include one of capacitance, resistance, or conductance.

The real-time DNA-based identity solution can be used as part of a multi factor authentication involving traditional solid state electronic semiconductor technology and nano-semiconductor technology. The solution described herein does not necessarily require DNA sequencing. Instead, the solution leverages the principles of physical chemistry, quantum mechanics, nano-semiconductor solid state electronics, and traditional semiconductor electronics.

Reference now is made in detail to the examples illustrated in the accompanying drawings and discussed below.

The DNA-based identity solutions described herein use information contained in a chromosomal DNA sample to verify the identity of a user. In particular, the solutions rely on chromosomes contained in the DNA sample, to verify users' identities. Chromosomes are giant molecules that contain the genetic codes of human beings. Each chromosome includes an ordered sequence of nucleotides which encodes various genes stored in the chromosome. The genetic code stored by each chromosome is composed of 4 nucleobases: adenine (A), cytosine (C), guanine (G), thymine (T). The sequence of bases forms part of the double helix structure commonly associated with genetic information. In addition to the ordered sequence of nucleotides, each chromosome includes enzymes, proteins, and other elements that provide structure to the chromosome and hold the base sequence together. For example, the chromosome includes magnesium (Mg²⁺), phosphate, ribose sugars, and the like.

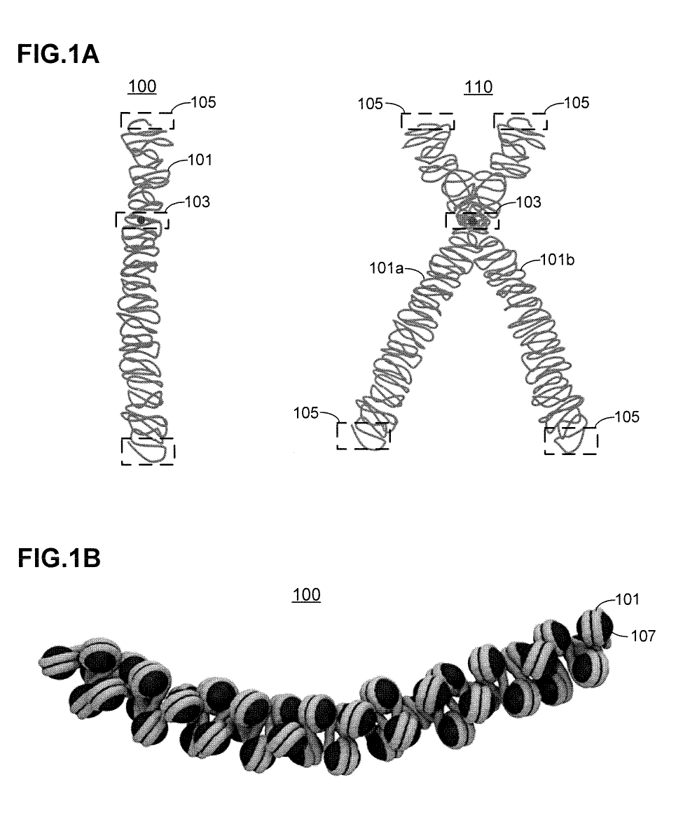

FIGS. 1A and 1B are diagrams illustratively showing a chromosome 100, which includes the giant molecule 101 forming a long double helix strand of DNA and storing the ordered sequence of nucleotides. The giant molecule 101 forming the double helix strand of DNA can be referred to as a chromatid. The giant molecule 101 is tightly wound and folded back onto itself, as shown in FIGS. 1A and 1B. In particular, histone proteins 107 create a scaffold that binds various parts of the molecule 101 to itself, thereby holding the molecule 101 in place so as to cause the molecule 101 to occupy less physical space. The particular position or configuration in which the molecule 101 is held by the histone proteins 107 is dependent on the genetic code stored by the chromosome. As such, a same chromosome, and/or copies of the chromosome storing substantially the same genetic code, is held by histone proteins 107 in a consistent structure, position, or configuration. The chromosome and copies of the chromosome storing substantially the same genetic code and genetic sequence are therefore bound or held in substantially the same configuration. As such, the chromosome and copies of the chromosome have the same structure (including histone structure), same size, and same characteristics. However, different chromosomes (i.e., chromosomes storing different genetic codes) are bound in structures having different configurations.

In general, the chromosome 100 is formed of a single double helix strand of DNA, as shown at 101. Each chromosome, however, can duplicate itself to form copies of the chromosome. For example, the chromosome may form a duplicate copy as part of a mitosis process taking place during cell replication (a process for creating a second copy of the cell). During the mitosis process, the chromosome may thus have two double helix strands of DNA 101a and 101b, as shown at 110. The two double helix strands of DNA store the same information as each other, and are thus substantially identical. The two double helix strands of DNA are attached together, to form the ‘X’ structure shown at 110, at a portion of the chromosome referred to as the centromere 103.

Each end of the chromosome is referred to as a telomere 105. The telomere stores a particular ordered sequence of nucleotides that is found at the end of the chromosome, and that can thus be used to locate or identify the end of the chromosome. The specific ordered sequence of nucleotides of the telomere protects the end of the chromosome from degradation. The TTAGGG sequence of the telomere is formed of three bases only (T, A, and G), and is the same for all humans.

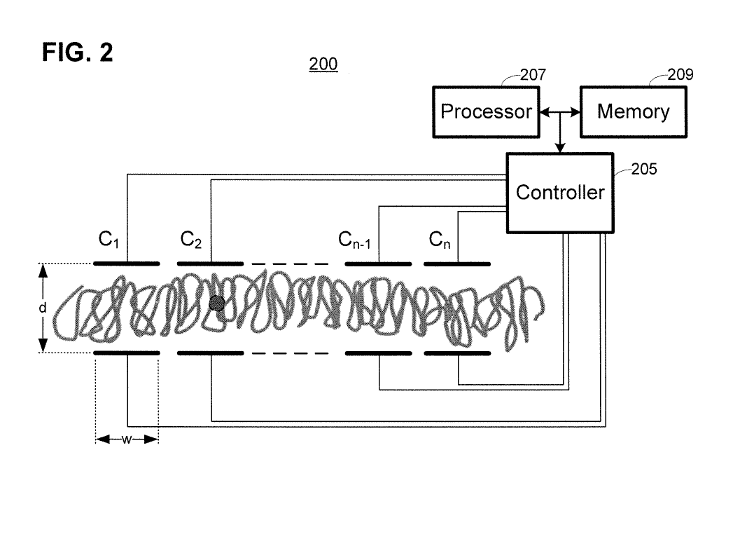

The DNA-based identity solution described herein relies on a chromosomal DNA sampler to obtain a chromosomal signature of a chromosome. The DNA sampler relies on a device similar to the device 200 shown in FIG. 2 for operation. Device 200 includes a linear array of capacitors C₁, C₂, . . . , Cₙ disposed end-to-end along a linear gap. In the example shown in the figure, the linear array includes n capacitors that each have a pair of capacitor plates. The plates of each capacitor are disposed on opposite sides of the linear gap. As a result, the linear gap extends, uninterrupted and substantially linearly, through device 200.

The plates of each pair are spaced apart from each other by a distance d. The distance d is selected so as to exceed the width of a chromosome disposed lengthwise in the linear gap. In one example, the distance d is selected to be approximately between 2 and 10 times the width of a chromosome. For example, for a chromosome width estimated to be 30 nm, the distance d may be selected to be greater than 30 nm (e.g., 31 nm, 50 nm, 90 nm, 150 nm, 300 nm, or the like). For a chromosome width estimated to be 700 nm, the distance d may be selected to be greater than 700 nm (e.g., 710 nm, 2.1 um, 3.5 um, 7 um, or the like).

Each capacitor has a width w, measured along the direction of the linear gap. The width w is selected to enable the chromosomal signature to be obtained. In one example, the width of the capacitor plate is in the range of 20-100 atom widths (e.g., 20-100 Angstroms (Å) long). In another example, the width w is selected to be between 2 and 100 times greater than the distance d. In yet another example, the width w is selected to be greater than or equal to the width of a DNA nucleotide (e.g., greater than or equal to 0.34 nm), and may more generally be selected in the range of the width of 10-100 DNA nucleotides (e.g., in the range of 3.4 nm-34 nm). The width w may alternatively be set based on the estimated length of a chromosome, and may be set to be between 1/100 and 1/1000 of the estimated length of a chromosome. All of the capacitors in the linear array generally have the same width as each other, although in some examples the capacitors may have different widths depending on their position within the linear array. Each capacitor is spaced apart from neighboring capacitors (i.e., from a previous and a next capacitor located along the linear gap) by a distance providing electrical isolation between the plates of the capacitor and the plates of the neighboring capacitors.

The device 200 further includes a controller 205 that is coupled to each of the capacitors in the array, and a processor 207 and memory 209 coupled to the controller 205. The controller 205 is operative to measure, for each capacitor in the array, an electrical property at the pair of capacitor plates. The electrical property can include one of capacitance, resistance, or conductance. As part of measuring the electrical property, the controller 205 may send a charge to a capacitor and measure the resulting voltage across the capacitor plates, so as to determine the capacitance of the capacitor. Alternatively or additionally, the controller may apply a current (or voltage) to the capacitor and measure the resulting voltage across (or current flowing through) the capacitor, so as to determine the resistance or conductance of the capacitor.

The processor 207 assembles the chromosomal signature of the chromosome based on the sequence of measurements performed by the controller 205 at each capacitor. The memory 209 stores the assembled chromosomal signature along with a plurality of other chromosomal signatures. Each chromosomal signature is stored in memory 209 in association with an identifier of the user having supplied the chromosome.

The electrical characteristics of a capacitor are dependent on the medium present between the capacitor's plates. For example, the capacitance of a capacitor varies depending on whether the space between the capacitor plates is filled with air, with a semiconductor, or with another appropriate material. Similarly, the electrical characteristics of each capacitor C₁-Cₙ of device 200 is dependent on the medium present between the capacitor plates. As such, the capacitance, resistance, conductance, or other electrical characteristic of each capacitor C₁-Cₙ varies depending on whether the space between the capacitor plates is filled with air, with a chromosome, or with another appropriate material. More particularly, the electrical characteristic of a capacitor varies depending on the particular chromosomal segment disposed between its plates, as detailed below.

As noted above in relation to FIGS. 1A and 1B, a chromosome includes histone proteins 107 that bind the giant molecule 101 of the chromosome into a particular configuration. The particular position or configuration in which the molecule 101 is held by the histone proteins 107 is dependent on the genetic code or sequence stored by the chromosome. As such, a same chromosome, and/or copies of the chromosome storing substantially the same genetic code, is held by histone proteins 107 in a consistent structure, position, or configuration. For example, the chromosome and copies thereof have the same structure, same dimensions (e.g., same distance between telomeres, or between a telomere and the centromere). However, different chromosomes and/or chromosomes storing different genetic codes are bound in structures having different configurations and potentially different dimensions. The chromosome's micro-structure (i.e., the genetic sequence encoded in the strand of DNA) thus determines the chromosome's macro-structure (i.e., the configuration into which the histone proteins 107 bind the molecule 101). In turn, a chromosome's macro-structure determines the particular amount and composition of matter present in any segment of the chromosome (i.e., a segment of length l along the length of the chromosome). For example, a chromosome segment (e.g., of length l) may include a tightly wound portion of the molecule having a high density of matter, while another chromosome segment (e.g., of a same length l) may include a loosely wound portion of the molecule have a low density of matter.

Because of the differences in structure along the length of a chromosome, the electrical characteristic of each capacitor C₁-Cₙ varies depending on the particular chromosomal segment disposed between its plates. As such, the electrical characteristic of each capacitor C₁-Cₙ varies depending on whether the chromosome segment located between the capacitor plates is tightly or loosely wound, or whether the chromosome segment has a similar or a different configuration as compared to another chromosome segment.

The device 200 can therefore determine a chromosomal signature for a chromosome by measuring, at each capacitor C₁-Cₙ located along the length of the chromosome mounted in the device 200, an electrical property at the capacitor. The sequence of measured electrical properties, ordered according to their position along the chromosome (and/or according to the position along the array of capacitors C₁-Cₙ), forms a chromosomal signature. Because chromosomes storing different genetic sequences have different micro-structures and therefore different macro-structures, chromosomes storing different genetic sequences also have different chromosomal signatures. Conversely, identical chromosomes have the same micro- and macro-structures, and therefore also have the same chromosomal signatures. Hence, the chromosomal signature measured by device 200 can be used to identify chromosomes, and determine whether two chromosomes encode the same genetic information (e.g., if the two chromosomes have identical chromosomal signatures) or different genetic information (e.g., if the two chromosomes have different chromosomal signatures).

The dimensions d and w of the capacitors C₁-Cₙ are selected so as to enable the controller 205 to obtain different chromosomal signatures from different chromosomes. For example, if the distance d or width w of capacitor plates are selected as being excessively large (e.g., a width that is approximately equal to the length of a chromosome), the controller 205 may obtain identical chromosomal signatures from different chromosomes and thus be unable to differentiate between different chromosomes. On the other hand, if the width of each capacitor plate is excessively small (e.g., a width that is approximately equal to several atoms), the controller 205 may not to be sensitive enough to sense changes in the electrical properties of the capacitor. Hence, the distance d and width w are selected to have intermediate values to enable a sufficiently unique chromosomal signature to be obtained from different chromosomes.

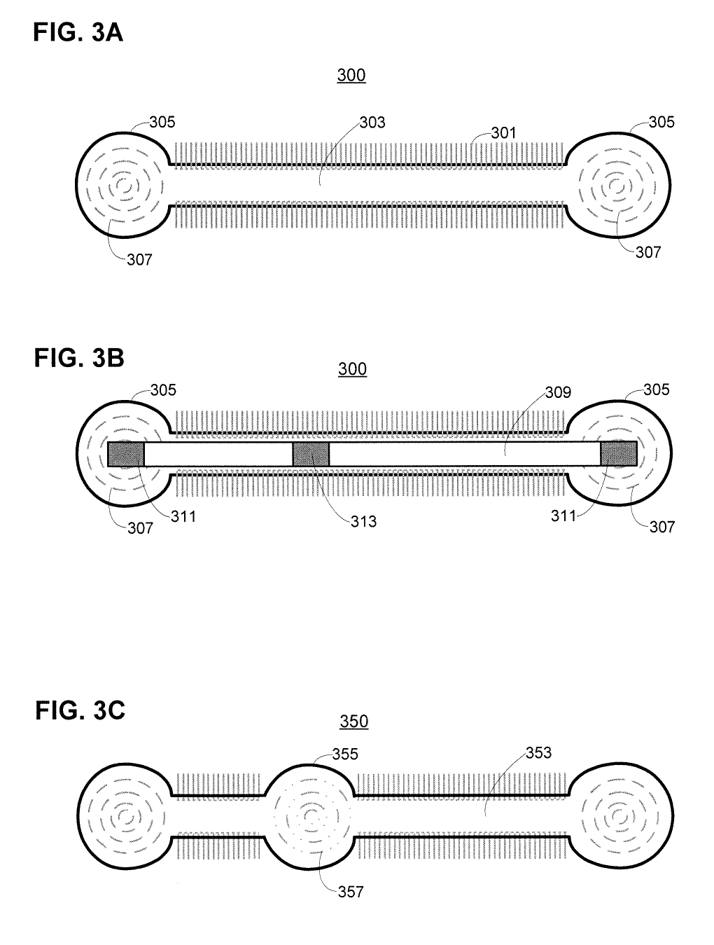

FIGS. 3A-3C are illustrative diagrams showing DNA samplers operative to obtain a chromosomal signature of a chromosome. The DNA sampler 300 of FIG. 3A includes a structure similar to the device 200 shown in FIG. 2. In particular, the DNA sampler 300 includes a linear array 301 of capacitors disposed on opposite sides of a linear gap 303. Each capacitor of the linear array 301 may be formed of nano wire(s), and the linear array 301 includes n capacitors disposed end-to-end along the gap 303. A controller (not shown) similar to controller 205 of FIG. 2 is coupled to each capacitor in the linear array 301, and is operative to measure an electrical property of each capacitor in the linear array 301. Additionally, the DNA sampler 300 includes one or more binding traps 305 operative to bind, attach to, or otherwise fix portions of chromosomes. The binding traps 305 may bind to chromosomes in order to fix the chromosome in the DNA sampler 300 such that the chromosome remains in the DNA sampler 300 while a chromosomal signature is obtained.

In the example shown in FIG. 3A, DNA sampler 300 includes two binding traps 305 that are disposed in alignment with the linear gap 303 at opposite ends of the linear gap 303. The binding traps 305 of DNA sampler 300 are telomere traps, each operative to bind with a telomere of a chromosome mounted in the DNA sampler to thereby fix the chromosome in the linear gap 303. In order to bind to chromosomes, each binding trap 305 may include a binding agent 307, such as a binding protein configured to bind to a particular segment of chromosome. In the example of FIG. 3A, the binding agent 307 may thus attract the telomeric sequence ends of a chromosome such that the chromosome's telomeres selectively bind into the binding traps 305 and the chromosome is thereby positioned in the linear gap 303.

In the case of telomere traps, the binding agent 307 can be a telomere binding protein that is configured to specifically or preferentially bind with telomeres, for example by binding a telomeric end based on structural attraction characteristics. Alternatively, the binding agent 307 may be a molecule that can react directly with a telomere and form a permanent bond. Cisplatin, for example, has specificity for reacting with guanines in the telomeric region of a chromosome to form permanent bonds with purine rings. The binding agent 307 may also be an antibody with the capacity to bind the telomeric end as an epitope.

The DNA sampler 300 has dimensions such that the length of the linear gap 303, measured in the example of FIG. 3A as the distance between the two binding traps 305, is substantially equal to the length of a chromosome designed to be mounted within the linear gap. In one example, a chromosome may have a length of approximately 280 um, and the DNA sampler 300 may have a linear gap 303 (and/or a distance between binding traps 305) set to 280 um. Other DNA samplers 300 may be sized for chromosomes of different sizes, and may in general have lengths within the range of 10-1,000 um.

The number of capacitors in the linear array 301 of DNA sampler 303 may be determined based on the dimensions of the DNA sampler 300. For example, the number of capacitors may be determined based on a ratio of the length of the linear gap 303 by the sum of the average width w of a capacitor in the array and the average distance between neighboring capacitors in the array. In another example, the number of capacitors in the linear array 301 is fixed (e.g., 100 capacitors, 1000 capacitors), and the average width w of each capacitor in the linear array 301 is determined based on the length of the linear gap 303 divided by the number of capacitors in the linear array 301.

FIG. 3B shows the chromosomal DNA sampler 300 having a chromosome 309 disposed within the sampler. As shown, the chromosome 309 is disposed in the linear gap 303, between each pair of capacitor plates of the capacitors of linear array 301. The chromosome 309 has a telomere 311 located at each end of the chromosome. Each telomere 311 is bound to a binding agent 307 of a binding trap 305 located at a respective end of the linear gap 303.

In addition to or instead of being bound to chromosomal DNA sampler 300 by telomeres, the chromosome 309 can be bound by other structures. For example, the chromosome can be found by its centromere 313. FIG. 3C shows an example of a DNA sampler 350 that includes a binding trap 355 disposed in alignment with the linear gap 353 and located at an intermediate point along the length of the linear gap 353. The binding trap 355 may be a centromere trap, that is configured to bind with the centromere of a chromosome (e.g., centromere 313 of chromosome 309) mounted in the DNA sampler 350. Similarly to binding trap 305, binding trap 355 includes a binding agent 357. Binding agent 357 may take any of the forms described above in relation to binding agent 307. In particular, binding agent 357 may be selected to bind to a centromere of a chromosome in order to fix the chromosome in the DNA sampler 350.

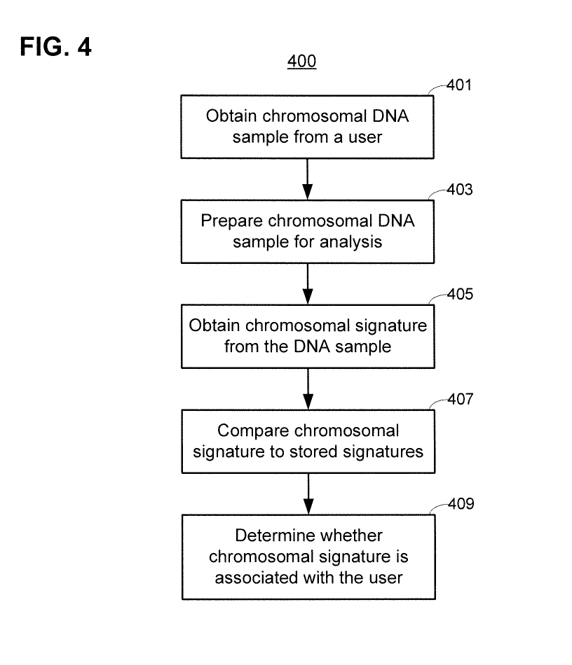

FIG. 4 is a flow diagram illustratively showing steps of a method 400 for verifying a user's identity in real-time using DNA-based information about the user. Method 400 begins with step 401, in which a chromosomal DNA sample is obtained from a user. The chromosomal DNA sample may be a sample including one or more cells of the user. The sample can be obtained, for example, by taking a swab inside the user's mouth, by obtaining one or more droplets of the user's blood, or through other appropriate means.

Once the chromosomal DNA sample is obtained, the DNA sample is prepared for analysis in step 403. The preparation can include steps for extracting chromosomes from the DNA sample, and causing at least one chromosome of the DNA sample to become mounted in a DNA sampler (such as DNA sampler 300). The preparation can thus include a step for performing cell lysis on the sample, in order to rupture or break down cell and nucleus walls in the chromosomal DNA sample to thereby release chromosomes from the nucleus. The cell lysis can be performed using one or more of optical, mechanical, acoustic, and electrical methods. At least one of the chromosomes can then be mounted in a chromosomal DNA sampler. In one example, the chromosomes are placed on a structure including one or more DNA samplers, and vibration is applied to the structure. The vibration causes the chromosomes to move around the structure, to bind with binding traps (e.g., binding trap 305), and to thereby become mounted in the linear gap of the DNA sampler.

In one example, as part of the process for causing a chromosome to become mounted in the chromosomal DNA sampler, a voltage is applied across the capacitors of the linear array of the chromosomal DNA sampler in order to assist in aligning the chromosome within the linear gap. The voltage is generally applied to all of the capacitors in the linear array, although in some examples different voltages can be applied to different ones of the capacitors. The application of the voltage can provide alignment of the chromosome in the electric field produced by the voltage in the linear gap.

Once a chromosome is mounted in the chromosomal DNA sampler, the chromosomal DNA sampler obtains a chromosomal signature from the chromosome in step 405. The chromosomal signature is obtained by measuring, for each pair of capacitor plates of the linear array having a portion of the chromosome disposed therebetween, an electrical property at the pair of capacitor plates. The sequence of measured electrical properties, ordered according to their position along the chromosome (and/or according to the position along the array of capacitors in the linear capacitor array of the DNA sampler), forms the chromosomal signature.

The chromosomal signature is based on one or more electrical properties including capacitance, resistance, conductance, or the like. In the case of capacitance, a controller of the chromosomal DNA sampler may apply a predetermined amount of electrical charge Q to a capacitor in the linear array, measure the resulting voltage V across the capacitor, and determine the capacitor's capacitance value C as the ratio of the amount of charge over the voltage: C=Q/V. In the case of resistance and/or conductance, the controller of the DNA sample can apply a predetermined current I to the capacitor, measure the resulting voltage V across the capacitor, and determine the resistance value R as the ratio of the voltage over the current: R=V/I. The controller may also obtain the resistance and/or conductance by applying a predetermined voltage V across the capacitor, measuring the resulting current I flowing through the capacitor, and determining the resistance value R as R=V/I. Once the electrical property of one capacitor of the array is determined, the processor can repeat the process on other capacitors in the array.

The chromosomal signature is compared, in step 407, to one or more other chromosomal signatures. Each of the other chromosomal signatures was previously obtained from a DNA sampler, associated with a particular user, and stored in a memory. In general, a user can be associated with multiple chromosomal signatures stored in memory, for example by being associated with chromosomal signatures associated with each of the user's chromosomes (e.g., in the case of most humans, 46 different chromosomes and associated signatures).

In one example, in step 407, a determination is made as to whether the chromosomal signature obtained in step 405 matches any of the other chromosomal signatures stored in memory. If a match is located, the identity of the user associated with the matched chromosomal signature is retrieved and used as part of the authentication process. In particular, in step 409, a determination is made as to whether the user seeking identity verification is the same user whose identity is associated with the matched chromosomal signature.

In another example, an identity of a user is obtained prior to performing step 407. For example, an identity of a user is obtained by prompting a user to enter a user identifier, to swipe a user identification card, or the like. As part of step 407, a determination is then made as to whether the chromosomal signature obtained in step 405 matches any of the chromosomal signatures stored in memory and associated with the identified user. If a match is located, the identity of the identified user is confirmed in step 409 and the confirmation can be used as part of the authentication process.

The method 400 for verifying a user's identity can include steps in addition to those shown in FIG. 4. In particular, the method 400 can be used as part of a multi-factor user identification/authentication process in which the DNA-based information is one of multiple factors used in authentication. In particular, as part of a multi-factor identification/authentication process, the user may be requested to provide a DNA sample as well as to provide a second authentication input such as one or more of a username, password, access card, fingerprint, iris scan, or the like. The multi-factor identification/authentication process may require that the DNA sample include a chromosome having a chromosomal signature matching a chromosomal signature that was previously obtained, stored, and associated with the user. The multi-factor identification/authentication process may additionally require that the second authentication input result in a match of credentials associated with that same user. If one or both of the DNA-based authentication and the second authentication input based authentication do not result in a match of credentials associated with the user, the user may be provided with a opportunity to repeat the identification/authentication process (e.g., by repeating one or both of the DNA-based authentication and the second authentication input based authentication). However, the number of repeat attempts may be limited. As such, if the user fails the identification/authentication process for the limited number of repeat attempts, the user may be blocked from further authentication attempts until a system administrator re-authorizes the user for access, or until a pre-determined time period expires (e.g., 1-day).

In some cases, information obtained as part of the DNA sample may be correlated with information obtained from the second authentication input. For example, information relating to a user's eye color is obtained through an iris-scan. The user's eye color, however, may also be encoded within the user's DNA, and information on the user's eye color may therefore also be obtainable by observing a particular pattern in the user's chromosomal signature. A correlation between information obtained from the iris-scan (or other second authentication input) and information obtained from the chromosomal signature can thus be used as part of the authentication process.

The examples described above use a linear array of capacitors to measure an electrical property at a plurality of locations along the length of a chromosome that is stationary within the linear array. In some examples, however, the measurement can be performed on a chromosome that is moving through the array. The chromosome may be moving in a linear direction along the length of the array, in a linear direction across the linear array, or rotating on itself within the array. In such examples, the chromosomal signature may include information on the rate of change of the electrical property at various locations along the length of the chromosome as the chromosome moves through the array at a know speed velocity.

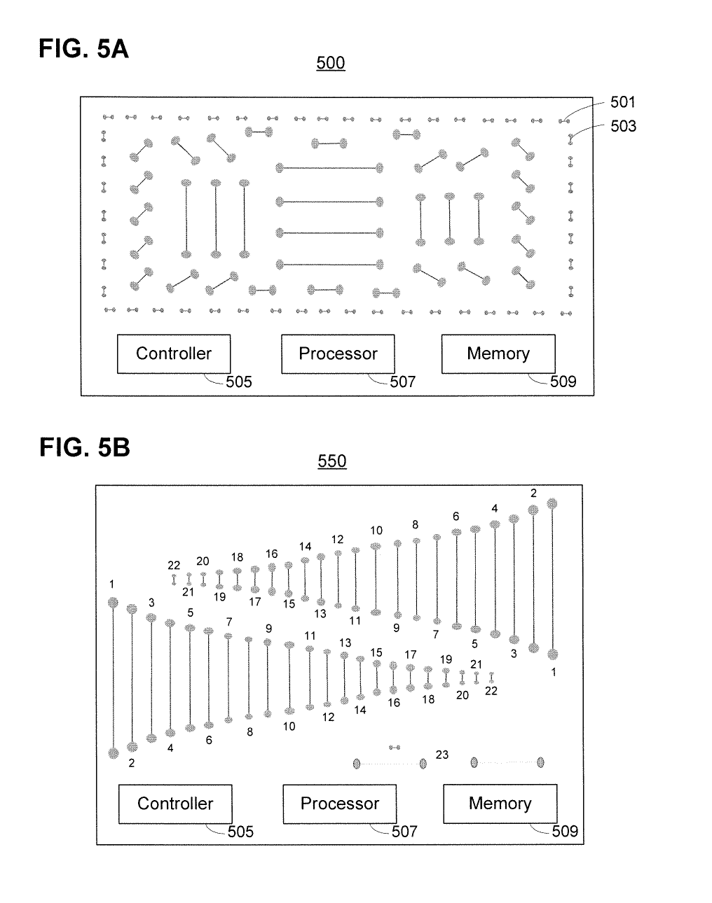

The DNA samplers 300 and 350 shown in relation to FIGS. 3A and 3B are DNA samplers configured to receive a single chromosome and obtain a chromosomal signature for the single chromosome. The DNA samplers 300 and 350 thus include only a single slot or linear gap for receiving a single chromosome. DNA samplers, however, may more generally be configured to receive multiple chromosomes. FIGS. 5A and 5B show illustrative DNA samplers 500 and 550 each configured to receive multiple chromosomes and to obtain chromosomal signatures for the multiple chromosomes. Each sampler may include one or more controller(s) 505/555, processor(s) 507/557, and memory(ies) 509/559 for performing functions similar to the controller 205, processor 207, and memory 209 described in relation to FIG. 2.

As shown in FIG. 5A, DNA sampler 500 includes multiple slots 501, 503 each configured to receive a single chromosome and obtain a chromosomal signature for the single chromosome. Each slot may thus include a linear array of capacitors disposed along a linear gap, and including binding traps. The capacitors of each slot are coupled to a controller 505 that is operative to measure an electrical property at the capacitor plates. Each slot may have its own controller 505, or a single controller 505 may be shared by multiple slots. Processor(s) 507 is coupled to the controller(s) 505, and is operative to obtain chromosomal signatures from the controller(s) 505. The processor(s) 507 is thus operative to obtain a chromosomal signature for any chromosome that is mounted in a slot of the reader. Memory(ies) 509 store chromosomal signatures in association with an identifier for a user associated with each signature. A chromosomal signature can be compared to other chromosomal signatures stored in memory(ies) 509 to determine whether the chromosomal signature matches any of the other signatures.

Slots may further be formed to have different widths, lengths, or configurations so as to be sized for different chromosomes. For example, as shown in FIG. 5A, slots can be formed to have a long, medium, or short length so as to preferentially bind long, medium, or short length chromosomes. The slots can further be formed to have various distances between binding traps (e.g., different distances between telomere traps, between a telomere trap and a centromere trap, or the like). The lengths/distances can be selected randomly, in order to maximize the probability that at least one slot of a DNA sampler 500 will readily bind a chromosome in a DNA sample.

The lengths/distances can alternatively be selected based on parameters of particular chromosomes, such as chromosome lengths and distances between chromosomes' centromeres and telomeres, in order to maximize the probability that the particular chromosomes will bind into the corresponding slots. For example, the DNA sampler 550 of FIG. 5B includes slots having lengths/distances selected based on parameters of human chromosomes. The DNA sampler 550 thus includes pairs of slots that are sized to correspond to sizes of each of the 22 pairs of chromosomes carried by humans and to the X,X or X,Y sex chromosomes respectively carried by female and male humans.

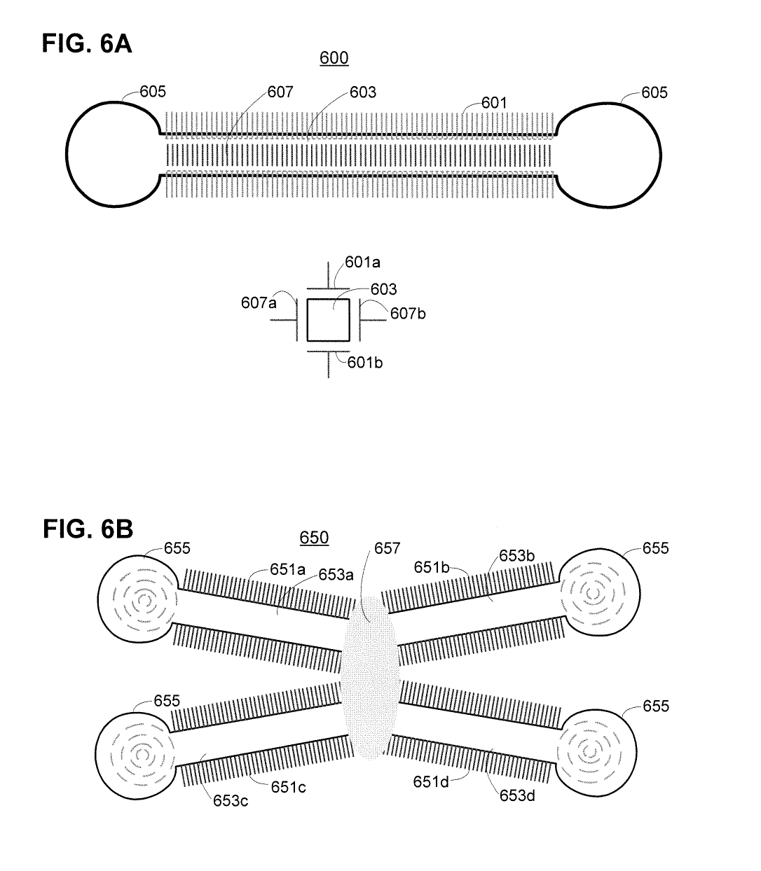

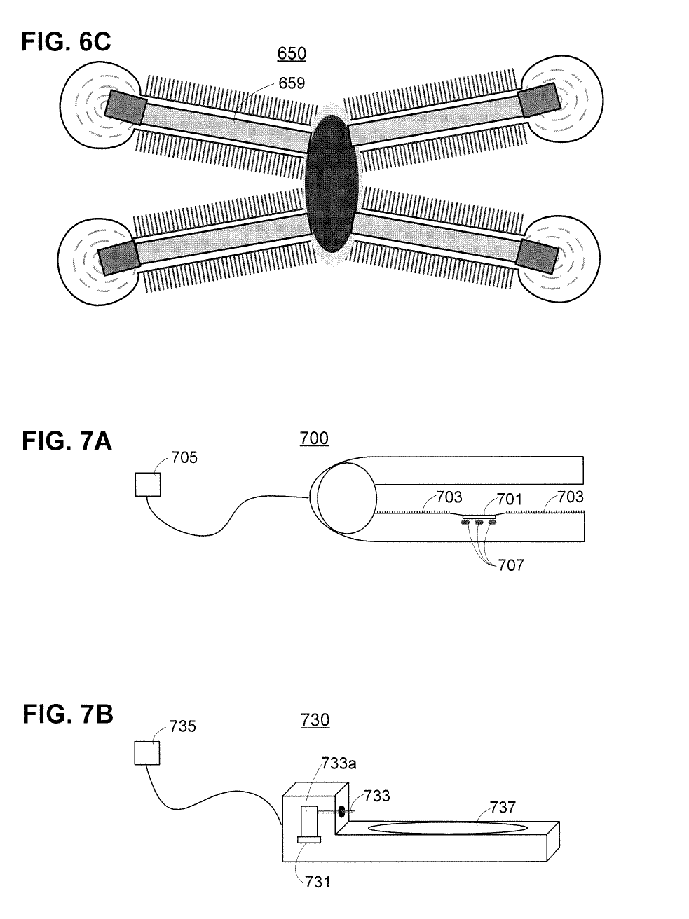

FIGS. 6A-6C show additional examples of DNA samplers.

FIG. 6A shows a DNA sampler 600 similar to the DNA sampler 300 of FIG. 3A. Similarly to DNA sampler 300, DNA sampler 600 includes a first linear array 601 of capacitors disposed along a linear gap 603, and binding traps 605. DNA sampler 600 further includes a second linear array 607 of capacitors disposed along the linear gap 603. Specifically, in the orientation shown in FIG. 6A, the first linear array 601 of capacitors includes capacitors having plates located on upper and lower sides of the linear gap 603. DNA sampler 600 further includes the second linear array 607 of capacitors that are orthogonal to the capacitors of linear array 601 and are disposed along left and right sides of the linear gap 603. Hence, as shown in the cut-away view shown in the lower portion of FIG. 6A, the first pair of capacitor plates 601a, 601b are disposed on a first set of opposite sides of the linear gap 603 (i.e., upper and lower sides of the linear gap 603), while the second pair of capacitor plates 607a, 607b are disposed on another set of opposite sides of the linear gap 603 (i.e., left and right sides of the linear gap 603).

Using the DNA sampler 600, two chromosomal signatures can be obtained from a single chromosome: a first chromosomal signature using the first linear array 601 of capacitors, and a second chromosomal signature using the second linear array 607 of capacitors. Alternatively, neighboring capacitors of the two linear arrays 601, 607 can be coupled in parallel and used to increase the DNA sampler's sensitivity to variations in chromosomal structure. For example, plates 601a and 607a can be coupled together while plates 601b and 607b can be coupled together, so as to form a single capacitor having larger plates. The single capacitor has a higher capacitance, and may thus have a higher change in capacitance depending on the particular chromosomal structure disposed between the plates.

FIGS. 6B and 6C show a DNA sampler 650 similar to the DNA sampler 300 of FIG. 3A, but configured to bind to a chromosome having two strands of DNA forming an X shape. Similarly to DNA sampler 300, DNA sampler 650 includes a first linear array 651a of capacitors disposed along a first linear gap 653a, and binding traps 655. The DNA sampler 650, however, includes multiple linear arrays 651a-d of capacitors, each array including capacitors disposed along a corresponding linear gap 653a-d. The linear arrays 651a-d and linear gaps 653a-d intersect in a region 657 configured to accommodate the centromere of a chromosome mounted in the DNA sampler 650. The region 657 can include a centromere trap, or the region 657 can be an open area within which the centromere can be accommodated.

FIG. 6C shows the DNA sampler 650 having a chromosome 659 disposed within the sampler. As shown, the chromosome 659 is disposed such that each DNA strand emanating from the centromere of the chromosome 659 is disposed in a respective linear gap 653a-d. The DNA sampler 650 can thus be used to obtain a chromosomal signature of an X-shaped chromosome 659 by obtaining chromosomal signatures of each DNA strand emanating from the centromere of the chromosome 659.

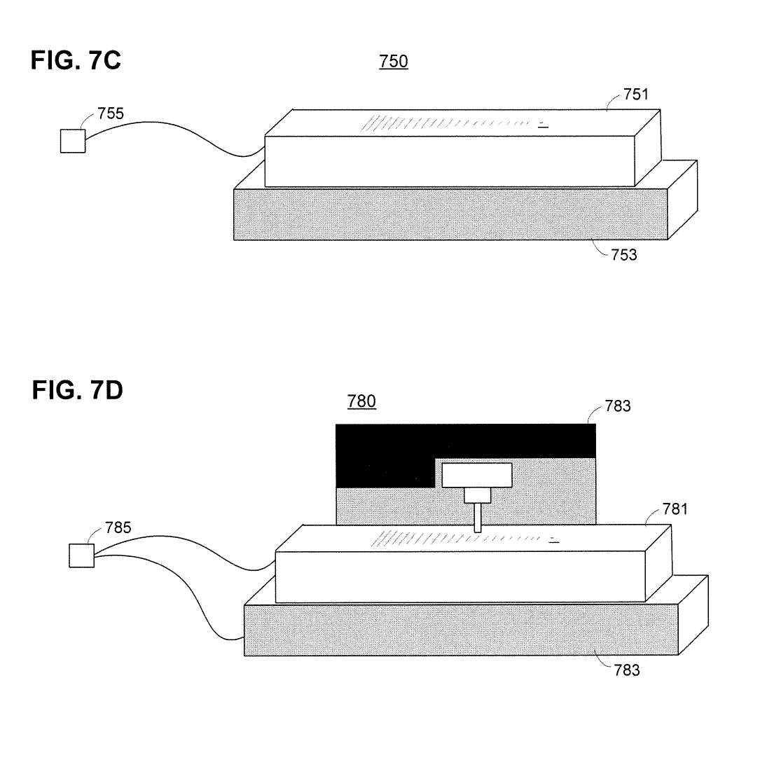

FIGS. 7A-7D show illustrative structures used to obtain and process DNA samples from users, such as structures that may be used to perform steps 401 and 403 of method 400 (as well as any of steps 405-409).

FIG. 7A shows an illustrative cheek/tongue clip structure 700 configured to clip onto a user's cheek tissue or tongue tissue to obtain a chromosomal DNA sample from the user. The structure 700 includes scrapers 703 operative to obtain the chromosomal DNA sample (e.g., a few cells of cheek or tongue tissue). The structure 700 further includes a DNA sampler 701, to which the chromosomal DNA sample is provided. DNA sampler 701 may be similar to any of the DNA samplers described previously, including any of DNA samplers 300, 350, 500, and 550. Additionally, one or more actuator(s) 707 such as piezoelectric actuator(s) is used to vibrate the structure so as to cause the chromosomal DNA sample to travel from the scrapers 703 to the DNA sampler 701, and to cause the chromosomes of the DNA sample disposed on the DNA sampler 701 to become mounted within slots of the DNA sampler 701. The clip structure 700 may further be configured to perform cell lysis, for example through optical, mechanical, acoustic, or electrical means. The structure 700 also includes a communication interface 705, such as a USB communication interface, used for connection to a computer or other system requiring user verification.

FIG. 7B shows an illustrative finger print scanner structure 730 configured to obtain both a finger print and a DNA sample from a user. The structure 730 includes a blood sampler 733 operative to obtain the DNA sample (e.g., a few droplets of blood) from the user. The structure 730 further includes a DNA sampler 731, to which the DNA sample is provided. DNA sampler 731 may be similar to any of the DNA samplers described previously, including any of DNA samplers 300, 350, 500, and 550. Additionally, additional sampling systems 733a may be provided to perform lysis on the DNA sample in order to release or extract chromosomes from the DNA sample. Cell lysis can be performed through optical, mechanical, acoustic, or electrical means. A finger print scanner 737 is used to obtain the user's finger print scan at the same time as the DNA sample is obtained. The finger print scan can be used with the DNA-based identity solution to perform a multi-factor verification of the user. The structure 730 further includes a communication interface 735, such as a USB communication interface, used for connection to a computer or other system requiring user verification.

FIG. 7C shows an illustrative structure 750 configured to process a DNA sample obtained from a user. The structure 750 includes a DNA sampler 751, such as DNA sampler 500 or 550, to which the DNA sample is provided. The structure 750 further includes a rocker assembly 753 operative to rock the DNA sampler 751 so as to cause the DNA sample deposited on the DNA sampler 751 (and chromosomes of the DNA sample) to move around the DNA sampler 751 and become mounted within slots of the DNA sampler 751. The structure 750 further includes a communication interface 755, such as a USB communication interface, used for connection to a computer.

FIG. 7D shows an illustrative structure 780 configured to process a DNA sample obtained from a user. The structure 780 includes a DNA sampler 781, such as DNA sampler 500 or 550, to which the DNA sample is provided. The structure 750 further includes an atomic force microscope 783 operative to cause chromosomes of the DNA sample to move around the DNA sampler 781 and become mounted within slots of the DNA sampler 781. The structure 780 further includes a communication interface 785, such as a USB communication interface, used for connection to a computer.

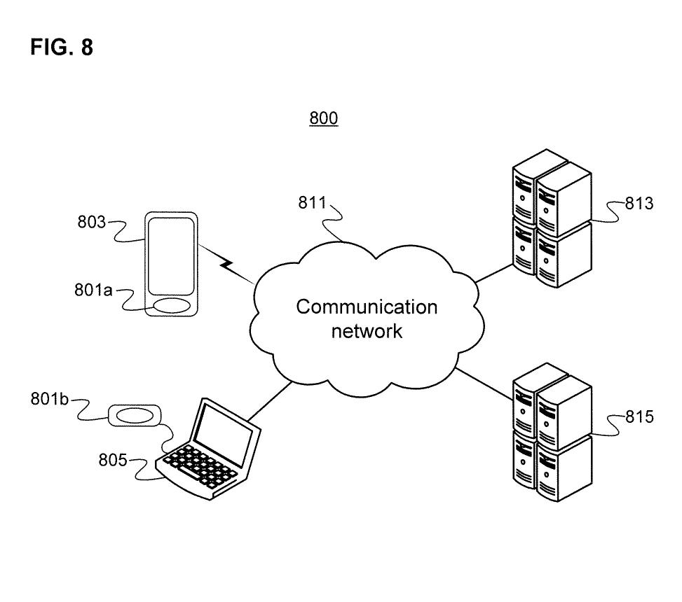

FIG. 8 shows a high-level functional block diagram of a system 800 of networks/devices that use chromosomal signatures for authentication. In the system 800, various types of DNA samplers are shown. A first DNA sampler 801a is embedded within a user device 803. A second DNA sampler 801b is a standalone device that can be communicatively connected to a user device 805 through a port of the user device 805 (e.g., a USB port). The DNA samplers 801a, b can be used to authenticate users attempting to access respective user devices 803, 805, and/or users attempting to access particular functions or programs run by the respective user device 803, 805 (e.g., a personal banking application). In some examples, the DNA samplers 801a, b have their own processors and memories controlling the samplers' operation. In other examples, however, at least some of the processing involved in the DNA-based authentication/identification process is performed by a processor and/or using a memory of the user device 803, 805.

In addition to controlling access to the user devices 803, 805, the DNA samplers 801a, b can be used to authenticate users attempting to access network services and applications available through the communication network 811. While communication network 811 is illustratively represented as a single network, the communication network may correspond to an interconnection of two or more networks including wired and/or wireless networks, public and/or private networks, mobile wireless network(s), the Internet and/or other packet data networks, or the like. The user devices 803, 805 may be configured for communication with the network 811 through a wired (as shown in the example of user device 805) or a wireless (as shown in the example of user device 803) communication link.

In networked examples, the DNA samplers 801a, b, may rely on network servers to complete the DNA-based authentication/identification process. In one example, the DNA samplers 801a, b operate in conjunction with an authentication server 815 communicatively coupled through the communication network 811. The authentication server 815 stores the chromosomal signature data for a plurality of users. Each chromosomal signature is stored in association with an identifier for a corresponding user. The authentication server 815 may additionally store other types of authentication information for the users, such as username and password combinations, device identity information, encryption/decryption keys, and the like. The DNA-based authentication process is performed using both the DNA samplers 801a, b and the authentication server 815.

For instance, a DNA sampler 801a, b may determine a chromosomal signature of a user from a DNA sample obtained from the user, and may provide the chromosomal signature to the authentication server 815 through the communication network 811. The chromosomal signature may be encrypted for transmission through the communication network 811, and may be transmitted in association with secondary authentication information in situations in which multi-factor identification/authentication is being performed. Upon receipt of the chromosomal signature, the authentication server 815 determines whether the chromosomal signature (and any secondary authentication information, if applicable) matches a chromosomal signature stored in the server. Upon determining a match, the authentication server 815 signals to the DNA sampler 801a, b, to the user device 803, 805, and/or to a network server 813 that the authentication is successful, so as to enable the authenticated user to access the user device 803, 805 and/or the network server 813 and network services. Upon failing to determine a match, the user is not granted access to the user device or to the network services.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

What is claimed is:

1. A DNA sampler device comprising:
a linear array of capacitors, in which pairs of capacitor plates are disposed end-to-end along a linear gap;
at least one binding trap structure, wherein the binding trap structure is disposed in alignment with the linear gap and is operative to bind with a chromosome mounted in the DNA sampler; and
circuitry coupled to each pair of capacitor plates of the linear array and operative to obtain a chromosomal signature of the chromosome mounted in the DNA sampler by measuring, for each pair of capacitor plates of the linear array having a portion of the chromosome disposed therebetween, an electrical property at the pair of capacitor plates.

2. The DNA sampler device according to claim 1, wherein a chromosome mounted in the DNA sampler is disposed in the linear gap with the pairs of capacitor plates located on opposing sides along a length of the chromosome.

3. The DNA sampler device according to claim 1, wherein the at least one binding trap structure is operative to bind with a centromere or a telomere of a chromosome so as to fix the chromosome in the linear gap.

4. The DNA sampler device according to claim 3, wherein the at least one binding trap structure comprises a protein, molecule, or antibody configured to bind with a portion of the chromosome.

5. The DNA sampler device according to claim 1, wherein the circuitry measures an electrical property selected from the group consisting of a capacitance, a resistance, and a conductance.

6. The DNA sampler device according to claim 1, wherein the circuitry comprises:
a controller coupled to each pair of capacitor plates of the linear array and operative to measure, for each pair of capacitor plates of the linear array, an electrical property at the pair of capacitor plates;
a processor coupled to the controller and operative to obtain a chromosomal signature of the chromosome based on a sequence of the measurements of the electrical property; and
a memory coupled to the processor and operative to store a plurality of chromosomal signatures each in association with a corresponding user identifier.

7. The DNA sampler device according to claim 1, wherein the circuitry is configured, for each pair of capacitor plates of the linear array having a portion of the chromosome disposed therebetween, to apply a predetermined amount of electrical charge Q to the capacitor plates, to measure a resulting voltage V between the capacitor plates, and to determine a capacitance value C as a ratio of the amount of electrical charge over the voltage: C=Q/V.

8. The DNA sampler device according to claim 1, wherein the circuitry is configured, for each pair of capacitor plates of the linear array having a portion of the chromosome disposed therebetween, to apply a predetermined current I to the capacitor plates, to measure a resulting voltage V between the capacitor plates, and to determine a resistance value R as a ratio of the voltage over the current: R=V/I.

9. A method comprising:
processing a chromosomal DNA sample obtained from a user in order to cause a chromosome of the chromosomal DNA sample to become mounted in a DNA sampler;
controlling the DNA sampler to obtain a chromosomal signature of the chromosome; and
determining whether the obtained chromosomal signature is associated with the user by comparing the obtained chromosomal signature to stored chromosomal signatures of chromosomes of the user.

10. The method according to claim 9, wherein:
the DNA sampler includes a linear array of capacitors in which pairs of capacitor plates are disposed end-to-end along a linear gap, and
the obtaining of the chromosomal signature includes measuring, for each pair of capacitor plates of the linear array having a portion of the chromosome disposed therebetween, an electrical property at the pair of capacitor plates.

11. The method according to claim 10, wherein the electrical property measured for each pair of capacitor plates in one of a capacitance, a resistance, and a conductance.

12. The method according to claim 9, wherein:
the DNA sampler includes a linear array of capacitors in which pairs of capacitor plates are disposed end-to-end along a linear gap, and
the processing the chromosomal DNA sample comprises causing the chromosome of the chromosomal DNA sample to be disposed in the linear gap with the pairs of capacitor plates located along a length of the chromosome.

13. The method according to claim 12, wherein:
the DNA sampler includes a binding trap structure disposed in alignment with the linear gap, and
the processing the chromosomal DNA sample comprises causing the chromosome of the chromosomal DNA sample to bind with the binding trap structure.

14. The method according to claim 13, wherein the binding trap structure comprises a protein, molecule, or antibody configured to bind with a portion of the chromosome.

15. The method according to claim 9, wherein the controlling the DNA sampler comprises, for each of a plurality capacitors of DNA sampler:
applying a predetermined amount of electrical charge Q to the capacitor;
measuring a voltage V across the capacitor resulting from the applying of the predetermined amount of electrical change; and
determining a capacitance value C of the capacitor as a ratio of the amount of electrical charge over the voltage: C=Q/V.

16. The method according to claim 9, wherein the controlling the DNA sampler comprises, for each of a plurality capacitors of DNA sampler:
applying a predetermined current I to the capacitor;
measuring a voltage V across the capacitor resulting from the applying of the predetermined current; and
determining a resistance value R of the capacitor as a ratio of the voltage over the current: R=V/I.
